(19) United States
(12) Patent Application Publication
(10) Pub. No.: US 2019/0328879 A1
(43) Pub. Date: Oct. 31, 2019

(54) CONSUMABLE ALCOHOLIC DRINK COMPOSITIONS CONTAINING AQUEOUS EMULSIFIED CANNABINOIDS

(71) Applicants: Rameshwar P. Ramcharran, Tarpon Springs, FL (US); Abdul Ally, Tarpon Springs, FL (US)

(72) Inventors: Rameshwar P. Ramcharran, Tarpon Springs, FL (US); Abdul Ally, Tarpon Springs, FL (US)

(21) Appl. No.: 15/964,891

(22) Filed: Apr. 27, 2018

(51) Int. Cl. A61K 47/10; A61K 31/05; A61K 31/352; A61K 9/107; A61K 9/08; C12G 3/04

(52) U.S. Cl. CPC A61K 47/10; A61K 31/05; C12G 3/04; A61K 9/107; A61K 9/08; A61K 31/352

(57) ABSTRACT

A composition that is absorbed by the human gastro intestinal tract including a combination of aqueous emulsified solution of cannabinoid, and alcohol. The aqueous emulsified solution of cannabinoid is made by evaporating a mixture of cannabinoid, polymeric carbohydrates, and polysaccharides to permit the cannabinoid to molecularly intercalate with the polymeric carbohydrates and mixing water with the mixture thereby creating a homogenous emulsion.

### BACKGROUND OF THE INVENTION

The present invention relates to consumable alcoholic drink compositions containing aqueous emulsified cannabinoids. More specifically, the present invention relates to a composition and method for making an alcoholic drink composition using cannabinoid aqueous emulsions to improve absorption of cannabinoid following ingestion.

Vodka has been produced since the early Middle Ages in Poland, and the first written mention of the word “vodka” was in 1405. Spirits including vodka were mainly used for medicinal purposes. Throughout the last 200 years, flavored vodkas have also been manufactured in many countries throughout the world. In Europe, vodkas flavored with herbs and fruits such as blackcurrants, cherry, green apple, lemon and watermelon are commonly available. According to Stefan Falimiertz in his 1534 treatise on herbs, vodka could serve to increase fertility and awaken lust. Early methodology for vodka manufacturing was simple, the initial distillation yielded low alcohol content and several distillations had to be carried out to yield a product that was 70% to 80% alcohol by volume (ABV) and subsequently diluted for consumption.

Towards the end of the 18th century, vodka was produced from various substances including carrots. High ABV concentration vodkas were typically supplemented with herbal alkaloids including cannabis and were used for medicinal purposes. Vodka today is made from grains including wheat, rye, corn or sorghum, but is also produced by fermentation of potatoes, barley, molasses, sugar and yeast. The practice of using 95% grain alcohol to make large quantities of vodka is seen in the US and Europe today.

Cannabinoids are a group of 21-carbon containing terpenophenolic compounds produced uniquely by Cannabis species. These plant-derived compounds may be referred to as phytocannabinoids. Although delta-9-tetrahydrocannabinol (THC) is the primary psychoactive ingredient, other known compounds with biological activity are cannabinol, cannabidiol (CBD), cannabichromene, cannabigerol, tetrahydrocannabivarin, and delta-8-THC. CBD, in particular, is thought to have significant analgesic, anti-inflammatory, and anxiolytic activity without the psychoactive effect (high) of delta-9-THC.

According to the National Institute on Drug Abuse (NIDA-NIH), the vast majority of research proposals received and funded by NIH on therapeutic benefits of cannabinoids have examined individual cannabinoid chemicals or, in a few cases, marijuana leaves delivered through some means other than smoking. Various factors make smoked marijuana less therapeutically promising than cannabinoid medications delivered through alternative routes of administration, including the potential harmful effects on the lungs and the risk for addiction. In addition, marijuana leaves contain numerous poorly understood chemicals in addition to THC and CBD. However, scientific study of the chemicals in marijuana, called cannabinoids, has led to two FDA-approved medications that contain cannabinoid chemicals in pill form. Research suggests that THC and other cannabinoids, may have potential in the treatment of pain, nausea, epilepsy, obesity, wasting disease, addiction, autoimmune disorders, and other conditions. Because the marijuana plant contains chemicals that may help treat a range of illnesses and symptoms, many people argue that it should be legal for medical purposes. In fact, a growing number of states have legalized marijuana for medical use in a number of disease states including epileptic seizures. In combination with alcohol, these marijuana based compounds can be absorbed by the human body more efficiently.

As can be seen, there is a need for consumable alcoholic drink compositions containing aqueous emulsified cannabinoids.

### SUMMARY OF THE INVENTION

In one aspect of the present invention, a composition for human consumption comprises alcohol in a liquid state combined with an aqueous emulsified solution of cannabinoid.

In another aspect of the present invention, a composition for human consumption consists of a liquid alcohol at about 30% up to about 42% by volume and an aqueous emulsified solution of cannabinoid at about 58% up to about 70% by volume.

These and other features, aspects and advantages of the present invention will become better understood with reference to the following drawings, description and claims.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention is a composition that is absorbed by the human gastro intestinal tract through a unique combination of an aqueous cannabinoid emulsion, and alcohol.

The composition includes the combination of alcohol with an aqueous emulsified solution of cannabinoid. The alcohol and the aqueous emulsified solution of cannabinoid includes about 30% up to about 42% alcohol by volume (ABV), and about 58% up to about to 70% aqueous emulsified solution of cannabinoid by volume. For example, the formulation includes about 40% alcohol by volume and 60% aqueous emulsified solution of cannabinoid by volume. Water may be further added for dilution of the alcohol to the desired percentage. The aqueous emulsified solution of cannabinoid remains in suspension in the vodka formulation.

The alcohol may be ethanol or ethyl alcohol in a liquid state, with a melting point of about negative (−) one hundred fourteen (114) degrees Celsius and above. For example, the alcohol may be vodka.

The aqueous emulsified solution of cannabinoid includes a mixture of glycoproteins, carbohydrates and cannabinoid. The aqueous emulsified solution of cannabinoid may include a concentration range from about 0.001 up to about 25 milligrams per milliliter of cannabinoid in the aqueous emulsion.

Cannabinoids are a group of chemical compounds that act upon the cannabinoid receptors in cells that are primarily in the brain which can include cannabinol, THC, or cannabidiol that is found in cannabis saliva (Marijuana) in the form of a phytocannabinoid (termed tetrahydrocannabinol or THC) being the psychoactive compound of cannabis or in the form of a synthetic cannabinoid.

In certain embodiments, the cannabinoid of the present invention is cannabidiol (CBD) and tetrahydrocannabinol (THC). Cannabidiol being the non-psychotropic cannabis constituent, may be derived from hemp in a liquid state, with a melting point of about sixty-six (66) degrees celsius and above. Hemp being defined as having less than about zero point three (0.3) percent % THC. Cannabidiol is a non-psychoactive cannabinoid that helps to avoid toxicity that is encountered with psychoactive cannabinoids. There are numerous cannabinoids that have been isolated from the cannabis plant. Tetrahydrocannabinol (THC) is the principal psychoactive constituent or cannabinoid of cannabis plants.

A method of making the invention may include the following. The composition of the present invention is made by first providing a cannabinoid in liquefied form, combining the cannabinoid with a mixture of polymeric carbohydrates and polysaccharides such as that found in powdered gum (such as naturally occurring gum arabic), heating the mixture to permit the cannabinoid to molecularly intercalate with the polymeric carbohydrates, creating a homogenous emulsion by the addition of water and gentle mixing. Next, the addition of alcohol is made to the emulsion to provide an alcohol content of about 30% to about 42%. The resulting vodka mixture facilitates preferential absorption through the gastro intestinal tract due to the combination of CBD and emulsifying agent by minimizing the normally hydrophobic nature of CBD.

It should be understood, of course, that the foregoing relates to exemplary embodiments of the invention and that modifications may be made without departing from the spirit and scope of the invention as set forth in the following claims.

What is claimed is:

1. A composition for human consumption comprising alcohol in a liquid state combined with an aqueous emulsified solution of cannabinoid.

2. The composition for human consumption of claim 1, wherein the alcohol is at 30% up to about 42% by volume and the aqueous emulsified solution of cannabinoid is at about 58% up to about 70% by volume.

3. The composition for human consumption of claim 1, wherein the alcohol is at about 40% by volume and the aqueous emulsified solution of cannabinoid is at about 60% by volume.

4. The composition for human consumption of claim 1, wherein the aqueous emulsified solution of cannabinoid remains in suspension in the alcohol.

5. The composition for human consumption of claim 1, wherein the alcohol is vodka.

6. The composition for human consumption of claim 1, wherein the aqueous emulsified solution of cannabinoid comprises a mixture of glycoproteins, polymeric carbohydrates, and cannabinoid.

7. The composition for human consumption of claim 6, wherein the cannabinoid is cannabidiol.

8. The composition for human consumption of claim 6, wherein the cannabinoid is a combination of cannabidiol and tetrahydrocannabinol.

9. The composition for human consumption of claim 6, wherein the aqueous emulsified solution of cannabinoid is made by evaporating the mixture to permit the cannabinoid to molecularly intercalate with the polymeric carbohydrates and mixing water with the mixture thereby creating a homogenous emulsion.

10. The composition for human consumption of claim 1, wherein cannabinoid is at a concentration range from about 0.01 up to about 10 milligrams per milliliter of the aqueous emulsified solution of cannabinoid.

11. A composition for human consumption consisting of a liquid alcohol at about 30% up to about 42% by volume and an aqueous emulsified solution of cannabinoid at about 58% up to about 70% by volume.
